SEQUENCE LISTING (10 sequences)

SEQ ID NO: 1 - DNA, 40 bp, SARS-CoV-2
aagaaggtgattgtgaagaagaagagtttgagccatcaac

SEQ ID NO: 2 - DNA, 40 bp, SARS-CoV-2
cttaaattccataatcaagactattcaaccaagggttgaa

SEQ ID NO: 3 - DNA, 40 bp, SARS-CoV-2
ttactcattcttacctggtgtttattctgttatttacttg

SEQ ID NO: 4 - DNA, 40 bp, SARS-CoV-2
gtgcagtgaaaagaacaatcaagggtacacaccactggtt

SEQ ID NO: 5 - DNA, 40 bp, SARS-CoV-2
atgtttgtttttcttgttttattgccactagtctctagtc

SEQ ID NO: 6 - DNA, 40 bp, SARS-CoV-2
ggaattctaacaatcttgattctaaggttggtggtaatta

SEQ ID NO: 7 - DNA, 40 bp, SARS-CoV-2
actctccaagggtgttcactttgtttgcaacttgctgttg

SEQ ID NO: 8 - DNA, 40 bp, SARS-CoV-2
tgaactgcaagatcataatgaaacttgtcacgcctaaacg

SEQ ID NO: 9 - DNA, 40 bp, SARS-CoV-2
acactagccatccttactgcgcttcgattgtgtgcgtact

SEQ ID NO: 10 - DNA, 40 bp, SARS-CoV-2
accccgcattacgtttggtggaccctcagattcaactggc

(19) United States
(12) Patent Application Publication
(10) Pub. No.: US 2022/0042944 A1
(43) Pub. Date: Feb. 10, 2022

(54) NANOCHANNEL SYSTEMS AND METHODS FOR DETECTING PATHOGENS USING SAME

(71) Applicant: PALOGEN, INC., Palo Alto, CA (US)

(72) Inventors: Bita Karimirad, Seoul (KR); Kyung Joon Han, Palo Alto, CA (US); Nazia Parveen, Islamabad (PK); Dongwon You, Seoul (KR)

(21) Appl. No.: 17/378,167

(22) Filed: Jul. 16, 2021

Related U.S. Application Data
(60) Provisional application No. 63/056,497, filed on Jul. 24, 2020.

(51) Int. Cl. G01N 27/447

(52) U.S. Cl. CPC G01N 27/4473; G01N 27/44773; G01N 27/44791

(57) ABSTRACT

A method of detecting a pathogen uses a 3D nanochannel device having top and bottom chambers, and a plurality of nanochannels. The method also includes functionalizing a nanochannel by coupling an oligonucleotide probe to an inner surface thereof. The method further includes adding a lysis buffer and patient sample to the top chamber. Moreover, the method includes extracting an oligonucleotide from the patient sample. In addition, the method includes placing top and bottom electrodes in the top and bottom chambers respectively and applying an electrophoretic bias therethrough. The method also includes applying a selection bias across first and second gating nanoelectrodes to direct flow of the oligonucleotide through the nanochannel. Moreover, the method includes applying a sensing bias through a sensing nanoelectrode. In addition, the method includes detecting an output current from the sensing nanoelectrode, and analyzing the output current from the sensing nanoelectrode to detect the oligonucleotide.

### CROSS-REFERENCE TO RELATED APPLICATION

This application claims priority to U.S. Provisional Application No. 63/056,497, filed on Jul. 24, 2020 under attorney docket number PAL.30010.00 and, entitled “NANOCHANNEL SYSTEMS AND METHODS FOR DETECTING PATHOGENS USING SAME,” the contents of which are hereby expressly and fully incorporated by reference in their entirety, as though set forth in full. This application includes subject matter similar to the subject matter described in co-owned U.S. Provisional Patent Application Ser. No. 62/566,313, filed on Sep. 29, 2017 under attorney docket number 165-101USIP and entitled “MANUFACTURE OF THREE DIMENSIONAL NANOPORE DEVICE”; U.S. Provisional Patent Application Ser. No. 62/593,840, filed on Dec. 1, 2017 under attorney docket number BTL.30002.00 and entitled “NANOPORE DEVICE AND METHOD OF MANUFACTURING SAME”; U.S. Provisional Patent Application Ser. No. U.S. Provisional Patent Application Ser. No. 62/612,534, filed on Dec. 31, 2017 under attorney docket number BTL.30003.00 and entitled “NANOPORE DEVICE AND METHODS OF ELECTRICAL ARRAY ADDRESSING AND SENSING”; U.S. Provisional Patent Application Ser. No. 62/628,214, filed on Feb. 8, 2018 under attorney docket number BTL.30004.00 and entitled “BIOMEMORY FOR NANOPORE DEVICE AND METHODS OF MANUFACTURING SAME”; U.S. Provisional Patent Application Ser. No. 62/711,234, filed on Jul. 27, 2018 under attorney docket number BTL.30005.00 and entitled “NANOPORE DEVICE AND METHODS OF DETECTING CHARGED PARTICLES USING SAME”; U.S. Utility patent application Ser. No. 16/147,362, filed on Sep. 26, 2018 under attorney docket number BTL.20001.00 and entitled “NANOPORE DEVICE AND METHOD OF MANUFACTURING SAME”; U.S. Utility patent application Ser. No. 16/237,570, filed on Dec. 31, 2018 under attorney docket number BTL.20003.00 and entitled “NANOPORE DEVICE AND METHODS OF ELECTRICAL ARRAY ADDRESSING AND SENSING”; U.S. Provisional Patent Application Ser. No. 62/802,459, filed on Feb. 7, 2019 under attorney docket number BTL.30004.01 and entitled “BIOMEMORY FOR NANOPORE DEVICE AND METHODS OF MANUFACTURING SAME”; U.S. Provisional Patent Application Ser. No. 62/826,897, filed on Mar. 29, 2019 under attorney docket number BTL.30006.00 and entitled “NANOPORE DEVICE AND METHODS OF BIOSYNTHESIS USING SAME”; U.S. Provisional Patent Application Ser. No. 62/874,766, filed on Jul. 16, 2019 under attorney docket number PAL.30007.00 and entitled “NANOPORE DEVICE AND METHODS OF DETECTING AND CLASSIFYING CHARGED PARTICLES USING SAME”; U.S. Utility patent application Ser. No. 16/524,033, filed on Jul. 27, 2019 under attorney docket number PAL.20005.00 and entitled “NANOPORE DEVICE AND METHODS OF DETECTING CHARGED PARTICLES USING SAME”; U.S. Provisional Patent Application Ser. No. 62/923,396, filed on Oct. 18, 2019 under attorney docket number PAL.30008.00 and entitled “NANOPORE DEVICE AND METHODS OF BIOSYNTHESIS USING SAME”; U.S. Provisional Patent Application Ser. No. 62/971,104, filed on Feb. 6, 2020 under attorney docket number BTL.30004.02 and entitled “BIOMEMORY FOR NANOPORE DEVICE AND METHODS OF MANUFACTURING SAME”; U.S. Provisional Patent Application Ser. No. 62/972,415, filed on Feb. 10, 2020 under attorney docket number PAL.30009.00 and entitled “NANOPORE DEVICE AND METHODS OF DETECTING AND CLASSIFYING CHARGED PARTICLES USING SAME”; and U.S. Utility patent application Ser. No. 16/832,990, filed on Mar. 27, 2020 under attorney docket number PAL.20006.00 and entitled “NANOPORE DEVICE AND METHODS OF BIOSYNTHESIS USING SAME.” The contents of the above-mentioned applications are fully incorporated herein by reference as though set forth in full.

### FIELD OF THE INVENTION

The present invention relates generally to point of care systems and devices and methods for detecting pathogens for infection diagnosis. In particular, the present invention relates to nanochannel sensors for point of care detections of pathogens, such as the SARS-CoV-2 coronavirus, by detecting specific target genome sequences.

### BACKGROUND

Severe Acute Respiratory Syndrome Coronavirus 2 (SARS-CoV-2), which causes Coronavirus Disease 2019 (COVID-19), is a member of the Coronaviridae family, genus Betacoronavirus. SARS-CoV-2 has been identified, and more than 187 million people have already been infected by various strains of this virus globally, resulting in significant morbidity and more than 4 million deaths. The World Health Organization (WHO) has declared the COVID-19 outbreak a pandemic and a public health emergency of international concern (PHEIC).

The list of reported symptoms of COVID-19 from sources such as the United States Centers for Disease Control and Prevention (CDC) is growing and evolving. A large number of varied symptoms have been reported from different groups of patients worldwide. COVID-19 symptoms can range from mild to severe disease. COVID-19 symptoms generally appear between 2 to 14 days after exposure to virus particles. Common symptoms of COVID-19 include cough, shortness of the breath, fever, and fatigue. Other symptoms such as headache, chills, muscle or joint aches, and sore throat can be seen in a number of patients. Impairment of taste and smell has also been reported. Liver enzyme abnormalities and a tendency to form blood clots may occur during infection. Patients with severe or critical disease often show evidence of a cytokine release syndrome (cytokine storm) with manifestations of progressive pneumonia, respiratory failure, kidney failure, or hypotension, frequently resulting in death.

The oropharyngeal and/or nasopharyngeal swab sample collection method recommended by the CDC retrieves samples from the respiratory mucosa at the backside of the nasal passage and throat (i.e., the nasopharynx). These may be placed in viral transport medium. Sample used for testing in clinical practice also include mid-turbinate samples, nasal washes, and nasal swabs (which are easier to obtain than nasopharyngeal swabs), sputum, and bronchoalveolar lavage fluid.

COVID-19 is caused by SARS-CoV-2, which belongs to a broad family of coronaviruses. Multiple strains of SARS-CoV-2 have been identified, but conserved sequences allow for the use of molecular diagnostic tools. SARS-CoV-2 is a positive-sense, single-stranded RNA virus with linear RNA. The virus size is about 60 to 200 nanometers. The full genome of SARS-CoV-2 is about 30,000 base pairs in length and has been sequenced from RNA extracted from patient samples. Polymerase chain reaction (PCR), using gene-specific nucleocapsid (N) and Open Reading Frame 1b (ORF1b) primers, has produced amplified overlapping PCR products covering the entire viral genome.

Leveraging low variability of the SARS-CoV-2 genomic RNA sequence, health authorities developed several nucleic acid tests to detect SARS-CoV-2 early in the course of the pandemic, and more recently, many academic and commercial laboratories have developed molecular tests, generally PCR-based, that vary in accuracy and sensitivity.

A SARS-CoV-2 molecular diagnostic that combines all of the features of extreme sensitivity, specificity, rapidity (minutes), robustness, reproducibility, low-cost, and high-throughput (or Point-of-Care [POC] deployment potential) represents the goal of molecular diagnostics development efforts. Given the severity of the illness, the importance of infection control and containment efforts, and the observation that viral shedding at lower titers may occur clinically (e.g., during the early/pre-symptomatic or late recovery phases of infection) it is imperative that an assay be as sensitive as possible. This will enable identification of individuals at either the beginning of infection or later in the course of disease, who may represent a source of spread to other individuals, and implementation of appropriate quarantine precautions. Assay performance is affected by many factors but, in the case of early or late infection, less sensitive molecular diagnostics may be especially prone to false negative results. In fact, overall false negative rates among existing molecular diagnostics vary, but may be as high as 30% or more. The WHO has already published information on several detection methods and established that the standard method of SARS-CoV-2 detection is real-time reverse transcription polymerase chain reaction (rRT-PCR), which is performed using samples from the nasopharyngeal and/or oropharyngeal swabs. One drawback of current testing methods is the long turnaround time (e.g., 30 minutes to two days or more), which reduces the clinical and public health utility of the information from the tests. The fastest is a point of care detection test that has cleared FDA Emergency Use Authorization (i.e., the Abbott ID NOW molecular SARS-CoV-2 test) has a turnaround time of approximately 5 minutes, but false negative results for 10% to 20% of samples may be seen, and have ranged from 6% to 48% in various reports (https://www.statnews.com/2020/05/15/fda-says-abbotts-5-minute-covid-19-test-may-miss-infected-patients/). More sensitive methods exist, such as digital PCR (Level of Detection 5-7 viral copies/microliter for crystal digital PCR per Stilla company representative presenting at Cambridge Health Institute Webinar 20 May 2020), but these require a significantly longer turnaround time. Most quantitative PCR assays take several hours to perform. Quantitative PCR is typically highly specific if probes are selected such that they bind to nucleotide sequences that do not bear significant homology to analogous sequences of other organisms.

The only method to determine whether detected SARS-CoV-2 virus is alive or dead is a costly cell culture method, which generates positive or negative cell culture results. However, cell culturing requires cell culture facilities and expert technicians to perform the cell culturing. Accordingly, it is not economically feasible to perform cell culturing for thousands of samples per day.

Accordingly, there is an immediate need for a highly sensitive, rapid, and accurate SARS-CoV-2 detection technique that can be used at a patient's bedside at early, middle, or late stages of infection, and to potentially screen for asymptomatic carriage or shedding among individuals with exposure to infected patients. This would allow for more timely diagnosis as well as better decisions about (a) the need to initiate isolation, and (b) the appropriate duration of isolation in the context of infection (of note, infected individuals may shed virus for several weeks). The above-described SARS-CoV-2 detection methods may be accurate and robust in a scientific laboratory setting; however, a significant amount of laboratory equipment and trained personnel are required to carry out almost all of these methods. In view of the current worldwide pandemic, there is an immediate need for a SARS-CoV-2 detection/screening test for everyday use by a large number of people, which rapidly produces highly sensitive, specific, robust, and reproducible results at low cost. In addition to the value conferred by its sensitivity throughout the time-course of infection (pre-symptomatic/early infection, mid-course and late infection), such an assay may also be useful in the setting in which a conventional quantitative PCR assay returns an ambiguous result.

Hybridization between two strands of DNA because of their complementary bases is one of the fastest methods in target sequence detection. Various methods have been used to perform such DNA based detection, including the use of labeled strands (i.e., primers or markers) designed to hybridize/anneal/bind with a particular known target sequence and to report the presence of the target sequence based on this binding. There are a number of methods for DNA sensing/detection, which use different techniques in combination with PCR and other nucleic acid amplification methods.

In molecular diagnostic techniques, an oligonucleotide probe is used to detect the virus via hybridization with viral genomic RNA circulating in the patient. The probe can increase the sensitivity (i.e., reduced false negatives) and specificity (i.e., reduced false positives) of the test. A properly designed oligonucleotide probe will bind properly to its complementary target (e.g., SARS-CoV-2 specific). When a probe does not bind to its target, the result may be a false negative and reduce the sensitivity of the test.

### SUMMARY

Embodiments described herein are directed to nanochannel based electrically assisted point of care platforms/detection systems with the potential use for central lab settings and methods of detecting SARS-CoV-2 using same. In particular, the embodiments are directed to various types (2D or 3D) of nanochannel based pathogen (e.g., SARS-CoV-2) detection systems, methods of using nanochannel array devices, and methods of detecting SARS-CoV-2 or other pathogens by using a nanochannel based 3D sensor system.

Embodiments of point of care devices, systems, and/or platforms for qualitative and quantitative detection of SARS-CoV-2 and COVID-19 respiratory infection in real-time are disclosed herein. Such devices, systems, and/or platforms are able to provide the isolation, extraction, and detection of the SARS-CoV-2 coronavirus in early stage by detection of the genomic RNA at very small concentrations. Extraction, preparation, and detection all are performed in a single chamber and the result is determined by reading electrical currents before and after adding the patient samples. A software system is operatively and communicatively coupled to the devices, systems, and/or platforms via wired (e.g., USB) and wireless (WiFi and/or Bluetooth) connection, and can display the results on a computer.

In one embodiment, a method of detecting a pathogen in a patient sample includes providing a 3D nanochannel device having top and bottom chambers, and a 3D nanochannel array disposed in the top and bottom chambers such that the top and bottom chambers are fluidly coupled by a plurality of nanochannels in the 3D nanochannel array. The method also includes functionalizing the 3D nanochannel array by coupling an oligonucleotide probe to an inner surface of the 3D nanochannel device defining the nanochannel, where the oligonucleotide probe is complementary to an oligonucleotide characteristic of the pathogen. The method further includes adding a lysis buffer to the top chamber. Moreover, the method includes adding the patient sample to the lysis buffer. In addition, the method includes extracting an oligonucleotide from the patient sample in the lysis buffer to form a sample solution. The method also includes placing top and bottom electrodes in the top and bottom chambers respectively. The method further includes applying an electrophoretic bias between the top and bottom electrodes. Moreover, the method includes applying a selection bias across first and second gating nanoelectrodes in the 3D nanochannel device to direct flow of the oligonucleotide through a nanochannel of the plurality of nanochannels. In addition, the method includes applying a sensing bias through a sensing nanoelectrode in the 3D nanochannel device. The method also includes detecting an output current from the sensing nanoelectrode. The method further includes analyzing the output current from the sensing nanoelectrode to detect the oligonucleotide.

In one or more embodiments, functionalizing the 3D nanochannel array by coupling the oligonucleotide probe to the inner surface of the 3D nanochannel device defining the nanochannel includes adding a solution of the oligonucleotide probe to the 3D nanochannel array, running a current through the 3D nanochannel array, washing the 3D nanochannel array, and reading a signal from the 3D nanochannel array to confirm functionalization of same. Washing the 3D nanochannel array may include using a microfluidic chamber.

In one or more embodiments, analyzing the output current from the sensing nanoelectrode to detect the oligonucleotide is performed by a processor coupled to the 3D nanochannel device. The processor may be coupled to the 3D nanochannel device via a wired connection. The processor may be coupled to the 3D nanochannel device via a wireless connection.

In one or more embodiments, detecting the 3D nanochannel device has more than 100 nanochannels therein. The 3D nanochannel device may include first, second, third, and fourth nanoelectrodes. The first nanoelectrode may be configured for sensing, and the second, third, and fourth nanoelectrodes may be configured for three dimensional sensing.

In one or more embodiments, extracting the oligonucleotide from the patient sample in the lysis buffer to form the sample solution includes heating the lysis buffer with the patient sample therein. The method may include displaying a qualitative result. The method may include displaying a quantitative result. The 3D nanochannel device may include a battery.

In one or more embodiments, the method can be carried out in a point of care, bedside system. The 3D nanochannel array may increase the surface area to volume ratio of the 3D nanochannel device. The method may be configured to detect the oligonucleotide at a 10 femtomolar concentration or less. The method may be configured to detect the oligonucleotide in about one minute.

In one or more embodiments, the method also includes functionalizing the 3D nanochannel array by coupling a second oligonucleotide probe to an inner surface of the 3D nanochannel device defining a second nanochannel. The second oligonucleotide probe is different from the first oligonucleotide probe, and the second oligonucleotide probe is complementary to a second oligonucleotide. The second oligonucleotide may be characteristic of the pathogen. The second oligonucleotide may be characteristic of another pathogen. The method may also include displaying first and second colors corresponding to number ranges for the oligonucleotide probe and the second oligonucleotide probe respectively. The method may also include displaying first and second plots corresponding to number ranges for the first oligonucleotide probe and the second oligonucleotide probe respectively.

In one or more embodiments, adding the patient sample to the lysis buffer includes disposing a swab with the patient sample thereof into the lysis buffer. The method may also include processing a single swab from a single patient. The method may also include processing a plurality of swabs from a plurality of patients using a plurality of 3D nanochannel arrays. The method may also include performing target genome sequencing using end-to-end barcode oligonucleotides or components thereof, which can be aligned on the inner surface defining the nanochannel and read.

In another embodiment, a 3D nanochannel device for detecting a pathogen in a patient sample includes a top chamber, a bottom chamber, and a 3D nanochannel array disposed in the top and bottom chambers such that the top and bottom chambers are fluidly coupled by a plurality of nanochannels in the 3D nanochannel array. The device also includes a probe coupled to an inner surface defining a nanochannel of the plurality of nanochannels, wherein the probe is complementary to a target molecule characteristic of the pathogen. The device further includes lysis buffer in the top chamber. Moreover, the device includes a top electrode disposed in the top chamber and a bottom electrode disposed in the bottom chamber. In addition, the device includes a first gating nanoelectrode electrically coupled to the nanochannel, a second gating nanoelectrode electrically coupled to the nanochannel, and a sensing electrode electrically coupled to the nanochannel. The device also includes a power source to apply an electrophoretic bias between the top and bottom electrodes, a selection bias across first and second gating nanoelectrodes in the 3D nanochannel device to direct flow of the target molecule through the nanochannel, and a sensing bias through the sensing nanoelectrode. The device further includes a sensor to detecting an output current from the sensing nanoelectrode. Moreover, the device includes a processor to analyze the output current from the sensing nanoelectrode to detect the oligonucleotide.

In one or more embodiments, the lysis buffer is configured to extract a genomic or protein component from the pathogen. The lysis buffer may be selected from the group consisting of common lysis buffer and deionize water. The probe may be an oligonucleotide probe, and the target molecule may be an oligonucleotide of the pathogen. The oligonucleotide probe in the nanochannel may have a c6amine, spacer, or bonding site in a 3′ or 5′ end thereof.

In one or more embodiments, the probe is a DNA aptamer probe, the target molecule is an antigen of the pathogen, and the DNA aptamer probe has an affinity for attachment to the antigen. The antigen may be selected form the group consisting of a spike protein or an M protein. The target molecule may be selected from the group consisting of DNA, RNA, mRNA, miRNA, antibody, antigen, and protein.

In one or more embodiments, the sensor detects changes in an electron transfer rate based on binding the target molecule triggering a change in a structure of the probe. The target molecule may be a charged biopolymer molecule that is negatively net-charged based on an isoelectric point and a zeta potential. The probe may be a bio-recognition receptor, the target molecule binding to the bio-recognition receptor may trigger an electrical signal, and the device may be an affinity-based sensor.

In one or more embodiments, the sensor is mounted on a digital and analog system, and the sensor is controlled through software, or by Bluetooth or USB connection to a computer or mobile phone. The lysis buffer may be deionized water, and the sensor may detect an output current due to a perturbation of water molecules, which causes field-induced movement actuation of the water molecules surrounding the probe and target molecule due to attachment of the target molecule to the probe, which perturbs electron transfer and affects the output current.

In one or more embodiments, the probe includes a plurality of different oligonucleotide probes, and each of the different oligonucleotide probes are complementary to a plurality of nucleotide targets in the pathogen. The probe may be configured to react with the target molecule in a bio-catalytic reaction. The probe may be an enzyme.

In one or more embodiments, the first gate electrode applies a positive gate bias, the second gate electrode applies a negative gate bias, and the positive and negative gate biases control a current inside of the nanochannel by controlling an ion carrier inside of the nanochannel. The positive gate bias applied by the first gating electrode may attract the negatively charge target in the nanochannel to facilitate hybridization of the probe and the target.

In one or more embodiments, the target molecule includes one gene sequence related to the pathogen. The device may also include a second probe coupled to a second inner surface defining a second nanochannel of the plurality of nanochannels. The second probe may be configured to detect a second target molecule of the pathogen. The second target molecule may be selected from the group consisting of DNA, RNA, a protein, an antibody, and an antigen.

In one or more embodiments, the device also includes a second probe coupled to a second inner surface defining a second nanochannel of the plurality of nanochannels. The second probe may be configured to detect a second target molecule of a second pathogen. The second target molecule may be selected from the group consisting of DNA, RNA, a protein, an antibody, and an antigen.

In one or more embodiments, the device is configured to perform target genome sequencing using the end-to-end barcode oligonucleotides or components thereof, which can be aligned on the inner surface defining the nanochannel and read.

In one embodiment, using a nanochannel device and system for detecting pathogens or charged biopolymers by using a biocatalytic component and molecules, which allow the system to electrically detect the reactivity of the analyte through a biorecognition molecule for instance enzymes and defining a nanochannel, includes a first gating nanoelectrode addressing a first end of the nanochannel. The device also includes a second gating nanoelectrode addressing a second end of the nanochannel opposite the first end.

In one embodiment, a nanochannel device has a plurality of nanochannel and a plurality of nanoelectrode embedded inside the nanochannel. The first electrode can apply positive gate bias and the second nanoelectrode can apply negative biases that can control the current inside the nanochannel by controlling the ion carrier inside the channel by using an AI control system.

In one embodiment, a nanochannel device has a plurality of nanochannel and a plurality of nanoelectrode embedded inside the nanochannel. The first electrode can apply positive gate bias to attract the negative charge pathogen-related DNA, RNA, miRNA, and/or mRNA, and target in the nanochannel and electrode surface area to facilitate hybridization of the probe and the target and second nanoelectrode can apply negative biases which can control the current inside the nanochannel by controlling the ion carrier inside the channel and vice versa.

In one embodiment, a nanochannel device and affinity-based sensors function at least partially based on the specific binding of the target analyte with a bio-recognition receptor and triggering the electrical signal in such a system and platform. Such a system and platform define a nanochannel, and include a first gating nanoelectrode addressing a first end of the affinity nanochannel. The device also includes a second gating nanoelectrode addressing a second end of the affinity nanochannel opposite the first end.

In one embodiment, a nanochannel device and system detects target DNA, RNA, mRNA, miRNA, antibody, antigen, protein, and etc. by measuring changes in electron transfer rate based on the binding of target molecules, which causes morphology changes and triggers changing the structure of the receptor strand and normal charge arrangement inside the nanochannel device.

In one or more embodiments, extracting the oligonucleotide from the patient sample includes heating the lysis buffer to about 98° C. to about 100° C. The method may include cooling the lysis buffer before applying the electrophoretic bias between the top and bottom electrodes.

In one or more embodiments, an inkjet technique or automatic pipettes is/are used for mechanically adding probes within individual pores or any similar method to functionalize and add probes within each nanochannel in the array of nanochannel can be used.

In another embodiment, a nanochannel device and system is configured to detect target DNA, RNA, mRNA, miRNA (micro RNA), antibody, antigen, protein, etc. of a target pathogen by measuring changes in electron transfer rate based on the binding of the target molecules to corresponding probes, which triggers changing the structure of the corresponding probes (e.g., receptor strands).

### BRIEF DESCRIPTION OF THE DRAWINGS

The drawings described below are for illustration purposes only. The drawings are not intended to limit the scope of the present disclosure. The drawings illustrate the design and utility of various embodiments of the present disclosure. It should be noted that the figures are not drawn to scale and that elements of similar structures or functions are represented by like reference numerals throughout the figures. In order to better appreciate how to obtain the recited and other advantages and objects of various embodiments of the disclosure, a more detailed description of the present disclosure will be rendered by reference to specific embodiments thereof, which are illustrated in the accompanying drawings. Understanding that these drawings depict only typical embodiments of the disclosure and are not therefore to be considered limiting of its scope, the disclosure will be described and explained with additional specificity and detail through the use of the accompanying drawings.

FIG. 1 generally and schematically depicts a method 100 for detecting a pathogen and diagnosing a disease according to some embodiments.

FIG. 2 is a graph illustrating a data analysis method according to some embodiments.

FIG. 3 is a graph illustrating the specificity and sensitivity of a method for detecting a pathogen and diagnosing a disease according to some embodiments.

FIGS. 4 and 5 schematically depict point of care pathogen detection systems/platforms according to some embodiments.

FIGS. 6A and 6B schematically depict flow cells for use in point of care pathogen detection systems/platforms according to some embodiments.

FIGS. 7A and 7B schematically depict a cartridge for use in point of care pathogen detection systems/platforms according to some embodiments.

FIG. 8 schematically depicts a main board for use in point of care pathogen detection systems/platforms according to some embodiments.

FIGS. 9-11 schematically depict hybridization of target oligonucleotides to complementary oligonucleotide probes in 3D nanochannel arrays according to some embodiments.

In order to better appreciate how to obtain the above-recited and other advantages and objects of various embodiments, a more detailed description of embodiments is provided with reference to the accompanying drawings. It should be noted that the drawings are not drawn to scale and that elements of similar structures or functions are represented by like reference numerals throughout. It will be understood that these drawings depict only certain illustrated embodiments and are not therefore to be considered limiting of scope of embodiments.

### DETAILED DESCRIPTION OF ILLUSTRATED EMBODIMENTS

Embodiments described herein are directed to nanochannel based electrically assisted point of care platforms/detection systems and methods of detecting SARS-CoV-2 using same. In particular, the embodiments are directed to various types (2D or 3D) of nanochannel based pathogen (e.g., SARS-CoV-2) detection systems, methods of using nanochannel array devices, and methods of detecting SARS-CoV-2 or other pathogens by using a nanochannel based 3D sensor system.

In some embodiments, a method of detecting a pathogen like SARS-CoV-2 (e.g., using a target oligonucleotide in the SARS-CoV-2 genome) includes providing a 3D nanochannel device having top and bottom chambers, and a 3D nanochannel array disposed in the top and bottom chambers such that the top and bottom chambers are fluidly coupled by a plurality of nanochannels in the 3D nanochannel array. Examples of such 3D nanochannel arrays are described in U.S. Provisional Patent Application Ser. Nos. 62/566,313, 62/593,840 and 62/923,396, and U.S. Utility patent application Ser. Nos. 16/147,362 and 16/524,033, the contents of which have been previously incorporated by reference herein. The method also includes functionalizing the 3D nanochannel array by coupling a target/pathogen specific oligonucleotide probe to an inner surface of the 3D nanochannel array defining the plurality of nanochannels, where the pathogen target oligonucleotide is complementary to the oligonucleotide probe.

The method further includes adding an oropharyngeal and/or nasopharyngeal swab to a buffer to form a solution including the isolated sample from the patient. The sampling method may follow the CDC approved method for oropharyngeal and/or nasopharyngeal swab sample collection. Moreover, the method includes adding the sample solution to the top chamber and gently mixing the sample solution with lysis buffer inside the top chamber. In addition, the method includes applying a current to the 3D nanochannel device for 5 minutes to bind the target oligonucleotide to the primer coupled to the walls of the plurality of nanochannels in the 3D nanochannel array.

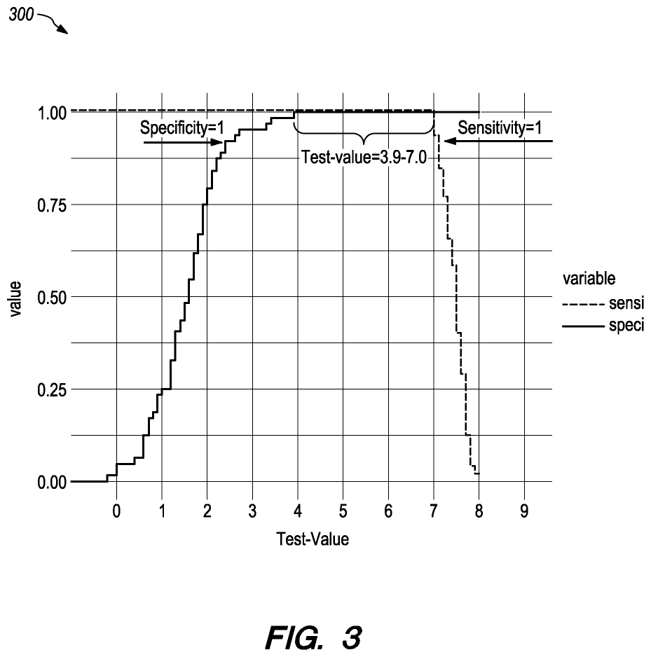

After binding the target oligonucleotide to the primer on the walls of the nanochannels, the nanochannels are washed by replacing the sample solution with deionized (DI) water and allowing the nanochannels to equilibrate for about one minute with an applied current. After washing the nanochannels with DI water for one minute, completeness of the washing can be determined by plotting an intensity graph of the applied current (see FIG. 3 and corresponding description).

The method also includes placing top and bottom nanoelectrodes in the top and bottom chambers respectively. The method further includes applying an electrophoretic bias between the top and bottom nanoelectrodes during a running mode and a sensing step. Moreover, the method includes applying a selection bias across first and second gating nanoelectrodes in the 3D nanochannel device to direct flow of the oligonucleotide through a nanochannel of the plurality of nanochannels.

In addition, the method includes applying a sensing bias through a sensing nanoelectrode in the 3D nanochannel device. The method also includes detecting an output current from the sensing nanoelectrode and processing the output current as a delta current in software operatively coupled to the 3D nanochannel device. The method further includes analyzing the output current from the sensing nanoelectrode to detect coupling of the pathogen specific oligonucleotide (e.g., a target oligonucleotide in the SARS-CoV-2 genome) and to determine the extent thereof. Electrically addressing and sensing individual nanochannel channels within multi-channel nanochannel arrays, is described in U.S. Provisional Patent Application Ser. No. 62/612,534 and U.S. Utility patent application Ser. No. 16/237,570, the contents of which have been previously incorporated by reference herein.

In some embodiments, the method may also include functionalizing the 3D nanochannel array by coupling a second oligonucleotide probe (complementary to a second region in a pathogen specific oligonucleotide) to an inner surface of the 3D nanochannel array defining a second nanochannel, where the second oligonucleotide probe is different from the first oligonucleotide probe. Analyzing the output current from the sensing nanoelectrode to detect and measure the pathogen specific oligonucleotide may include comparing the output current and the sensing bias to corresponding values in a reference table. Analyzing the output current from the sensing nanoelectrode to detect coupling of the oligonucleotide may include using an effect of a negative charge in a phosphate backbone of the oligonucleotide. Charge carriers in the 3D nanochannel device may include DI water, H+ ions, and OH− ions.

In some embodiments, the pathogen specific oligonucleotide is an SARS-CoV-2 RNA fragment. In other embodiments, the pathogen specific oligonucleotide may be an RNA or DNA fragment from other pathogens. In some embodiments, the pathogen specific oligonucleotide may be extracted from patient samples such as cell free DNA, tissue, cell culture medium, nasal swab, nasal wash, mid-turbinate swab, sputum, bronchoalveolar lavage fluid, serum, urine, plasma, or saliva inside the top chamber of the 3D nanochannel device by disposing the patient sample in lysis buffer and heating the lysis buffer to 98° C. for several minutes.

Exemplary nucleic acid sequences for use with the 3D nanochannel devices and pathogen detection methods described herein are listed in the Table 1. The nucleic acid sequences were present in Coronavirus samples taken from COVID-19 patients in China, the United States of America (CA, MA, WI, AZ, and IL), Nepal, Sweden, Australia, Hong Kong, Taiwan, and Korea. The present sequences are designed by the inventors, from the approved sequences derived after sequencing and the region which have been confirmed by the CDC for SARS-CoV-2 detection and COVID-19 diagnosis. Note that the list below is not comprehensive, and that this invention subsumes other probes specific for COVID-19, or other viruses, that may accurately enable molecular detection.

In one embodiment a device and system for detecting pathogen includes one gene sequence as a probe to capture the target molecule, which relates to a specific pathogen. Accordingly, the device and system can have more than one type of probe to capture one gene and detect the pathogen genome which may be DNA, RNA, protein, antibody, antigen, and relate to the particular pathogen. For instance, the target fragment and or probe can detect a wide range (e.g., millions) of DNA, RNA, and/or protein targets, which derive from one or different pathogens.

In one embodiment such device and system can operate target genome sequencing by using the end to end barcode oligonucleotides or component which can be aligned after the reading and electrical scanning the sensor surface. Exemplary oligonucleotide probes for use with SARS-CoV-2 detection platforms are listed in Table 1.

TABLE 1
Oligonucleotide Probes for SARS-CoV-2 Detection Platforms

| | | | | Genes |
|---|---|---|---|---|
| | | | | corresponding |
| | | | | to probe |
| Sr# | Start | End | 40 bp probe | sequence |
| seq1 | 3060 | 3099 | AAGAAGGTGATTGTGAAGAAGAAGAGTTTGAG | RdRP/ORF1 |
| | | | CCATCAAC | ab |
| seq2 | 1093 | 1133 | CTTAAATTCCATAATCAAGACTATTCAACCAAG | RdRP/ORF1 |
| | | | GGTTGAA | ab |
| seq3 | 9556 | 9596 | TTACTCATTCTTACCTGGTGTTTATTCTGTTATT | RdRP/ORF1 |
| | | | TACTTG | ab |
| seq4 | 10974 | 11014 | GTGCAGTGAAAAGAACAATCAAGGGTACACAC | RdRP/ORF1 |
| | | | CACTGGTT | ab |
| seq5 | 21563 | 21603 | ATGTTTGTTTTTCTTGTTTTATTGCCACTAGTCT | RdRP/ORF1 |
| | | | CTAGTC | ab and S |
| seq6 | 22869 | 22909 | GGAATTCTAACAATCTTGATTCTAAGGTTGGTG | S |
| | | | GTAATTA | |
| seq7 | 25608 | 25648 | ACTCTCCAAGGGTGTTCACTTTGTTTGCAACTT | ORF3a |
| | | | GCTGTTG | |
| seq8 | 27851 | 27891 | TGAACTGCAAGATCATAATGAAACTTGTCACG | ORF7b |
| | | | CCTAAACG | |
| Seq9 | 26332 | 26372 | ACACTAGCCATCCTTACTGCGCTTCGATTGTG | E gene |
| | | | TGCGTACT | |
| Seq10 | 28309 | 28349 | ACCCCGCATTACGTTTGGTGGACCCTCAGATT | N gene |
| | | | CAACTGGC | |

The 3D nanochannel devices and platforms described herein can be incorporated in an automated point of care pathogen detection system leveraging microfluidics for preparation and extraction of patient samples, pathogen detection via oligonucleotide hybridization, and data analysis. Such point of care pathogen detection systems can be used to detect the SARS-CoV-2 virus and diagnose COVID-19 disease using a bedside point of care system. Alternatively, samples may be appropriately collected and forwarded, under suitable conditions and within a specified timeframe, to a CLIA certified laboratory at which testing may be conducted.

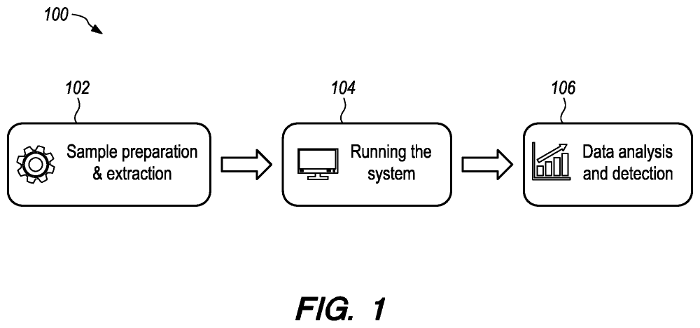

FIG. 1 depicts three general steps in a method 100 for detecting a pathogen (e.g., SARS-CoV-2) and diagnosing a disease (e.g., COVID-19) according to some embodiments. At step 102, patient samples are collected, prepared, and target oligonucleotides are extracted therefrom as described herein. At step 104, the extracted patient sample is run on a point of care system including the 3D nanochannel device to generate data relating to the target oligonucleotides as described herein. At step 106, the generated data is analyzed to detect the oligonucleotides in the patient sample for pathogen detection and disease diagnosis as described herein. Software allowing data analysis and representation has been validated and verified.

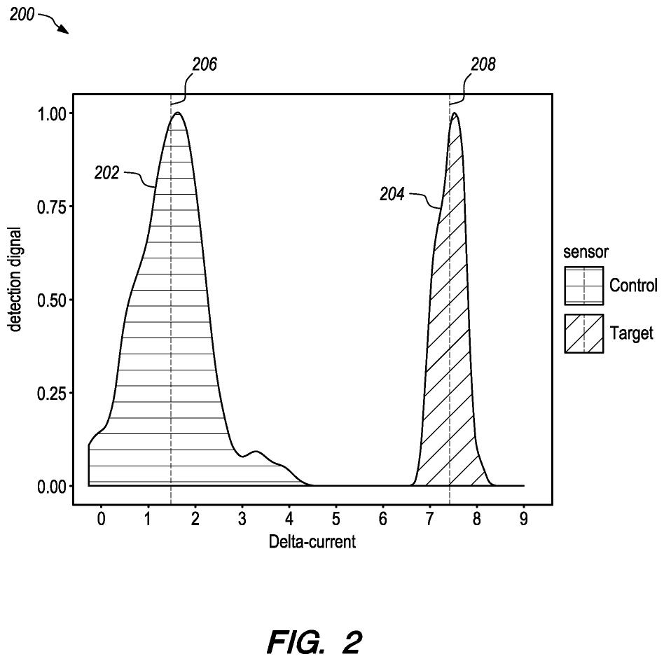

FIG. 2 is a graph 200 illustrating a data analysis method for detecting SARS-CoV-2 specific oligonucleotides and diagnosing COVID-19 disease using 3D nanochannel devices according to some embodiments. The 3D nanochannel devices detect an oligonucleotide detection signal in response to an applied delta current. The graph 200 plots a detection signal (Y axis) vs. an applied delta current (X axis) for a control sample 202 and a sample containing SARS-CoV-2 specific oligonucleotides (target) 204. The amount of applied delta current to generate the control and target curves 202, 204 and their normal distributions across respective mean values 206, 208 can be used to distinguish a positive sample 204 from a negative sample 202.

FIG. 3 is a graph 300 illustrating the specificity and sensitivity of a method for detecting SARS-CoV-2 specific oligonucleotides and diagnosing COVID-19 infection using 3D nanochannel devices according to some embodiments. In FIG. 3 test results are plotted along the X-axis while specificity and sensitivity for each test result are plotted along the Y-axis. The graph 300 shows that both the specificity and sensitivity of the method are each about 100% for test values between about 3.9 and about 7.0.

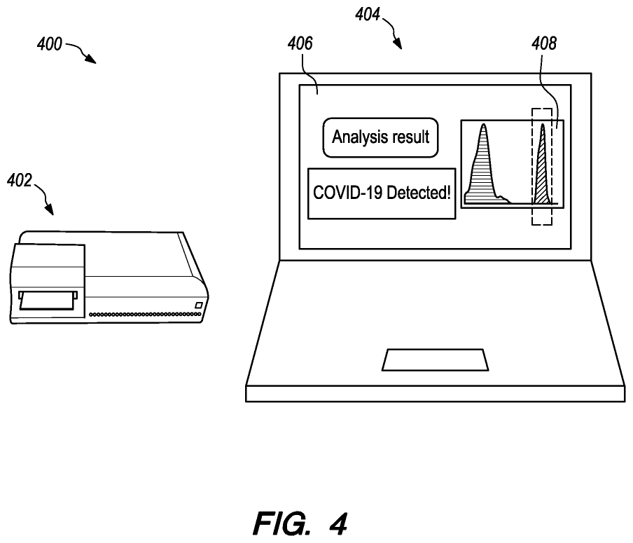

FIG. 4 schematically depicts a point of care pathogen (e.g., SARS-CoV-2) detection system/platform 400 including a 3D nanochannel device according to some embodiments. The system 400 includes a small footprint 3D nanochannel detection device 402 to analyze patient samples and generate data, and a computer 404 with a processor programmed to analyze the generated data as described herein. The input to the system may be a patient swab. The output of the system 400 may be a Graphical User Interface 406 showing a graph 408 and an indication of whether the pathogen has been detected.

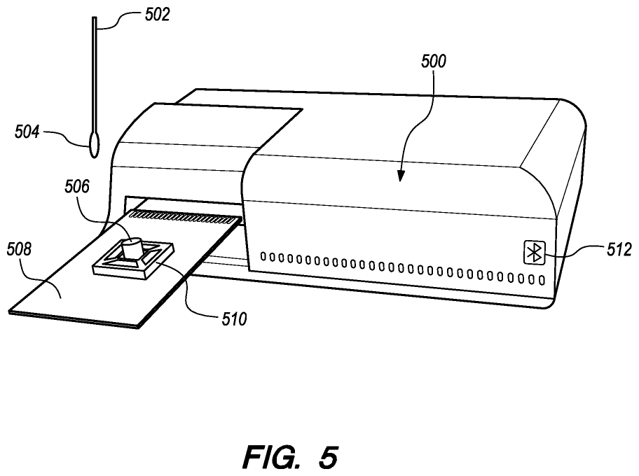

FIG. 5 schematically depicts a point of care pathogen (e.g., SARS-CoV-2) 3D nanochannel detection device 500 according to some embodiments. The device 500 has a small footprint for use at a point of care/bedside. The device 500 includes a 3D nanochannel cartridge 508, which includes a flow cell 510 having top and bottom chambers, and a 3D nanochannel array disposed in the top and bottom chambers such that the top and bottom chambers are fluidly coupled by a plurality of nanochannels in the 3D nanochannel array, as described herein. The cartridge 508 may be disposable and may minimize or eliminate contamination between patient samples. The cartridge 508 may also increase the throughput of the device 500 by minimizing cleaning requirements. The device 500 may be used with a swab 502 with a patient sample on a tip 504 thereof as described herein. The patient sample on the swab tip 504 may be loaded into a top chamber 506 of the flow cell 510 of the device 500 as described herein. After processing and analysis, generated results may be transmitted to a computer (see FIG. 4) via a Bluetooth connection 512.

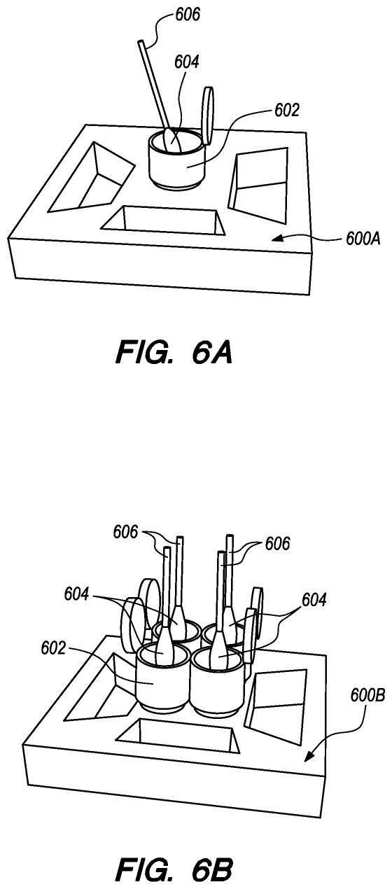

FIGS. 6A and 6B schematically depict flow cells 600A, 600B for use in point of care pathogen (e.g., SARS-CoV-2) 3D nanochannel detection devices according to some embodiments. The flow cells 600A, 600B may be used with the point of care pathogen (e.g., SARS-CoV-2) 3D nanochannel detection systems and devices 400, 500 depicted in FIGS. 4 and 5. The flow cells 600A, 600B have one (600A) or more (e.g., four in 600B) sample containers 602 with lysis buffer therein. A patient sample on a tip 604 of a swab 606 may be loaded into a sample container 602 containing a sample solution (i.e., patient sample in lysis buffer) therein for solubilization of the patient sample. The sample container 602 may be fluidly coupled to a top chamber of the flow cell 600A, 600B to deliver the patient sample to the 3D nanochannel device therein (see FIG. 5). The flow cell 600A has one sample container 602, and the flow cell 600B has four sample containers. The flow cells 600A, 600B can process patient samples from one patient at a time (see FIG. 6A) or from a plurality of samples from a plurality of patients (see FIG. 6B) using a plurality of isolated 3D nanochannel arrays in one system.

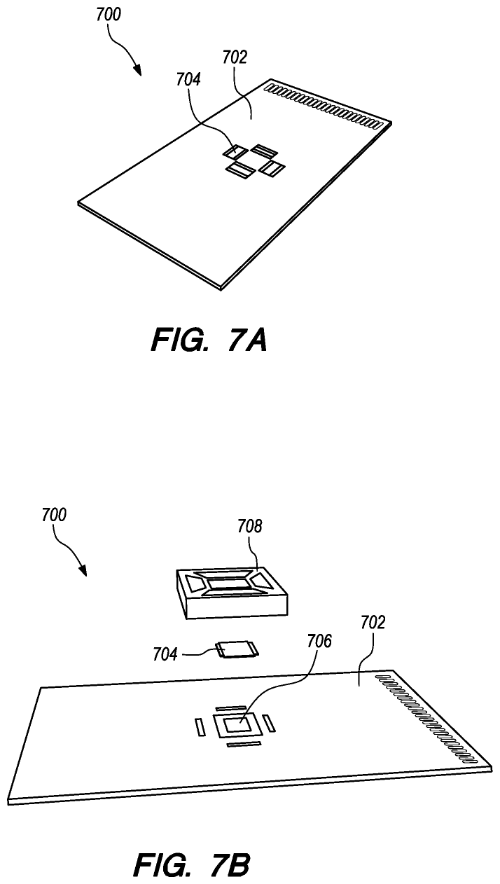

FIGS. 7A and 7B schematically depict a cartridge 700 for use in point of care pathogen (e.g., SARS-CoV-2) 3D nanochannel detection devices according to some embodiments. The cartridge 700 includes a cartridge/sensor board 702 and a 3D nanochannel device/sensor 704 coupled to the cartridge/sensor board 702 with an attachment/wiring harness 706. A flow cell 708 is attached to the 3D nanochannel device/sensor 704 and the cartridge/sensor board 702. During manufacturing, the cartridge/sensor board 702 can be first formed. Next, the wiring harness 706 can be attached thereto or formed thereon. Next, the 3D nanochannel device/sensor 704 can be attached to the wiring harness 706. Next, the flow cell 708 can be attached to the 3D nanochannel device/sensor 704 and cartridge/sensor board 702.

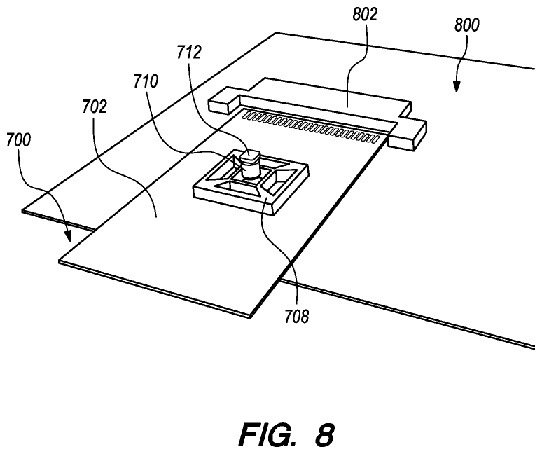

FIG. 8 schematically depicts a main board 800 for use in point of care pathogen (e.g., SARS-CoV-2) 3D nanochannel detection devices according to some embodiments. The main board 800 is a part of point of care pathogen (e.g., SARS-CoV-2) 3D nanochannel detection devices, such as the point of care pathogen (e.g., SARS-CoV-2) 3D nanochannel detection system and device 400, 500 depicted in FIGS. 4 and 5. The main board 800 includes a cartridge connector 802 configured to electrically and physically couple a cartridge 700 thereto. The cartridge 700 includes a cartridge/sensor board 702, a flow cell 708, a sample container 710, and a heating system 712. The heating system 712 may be used to process the patient sample as described herein.

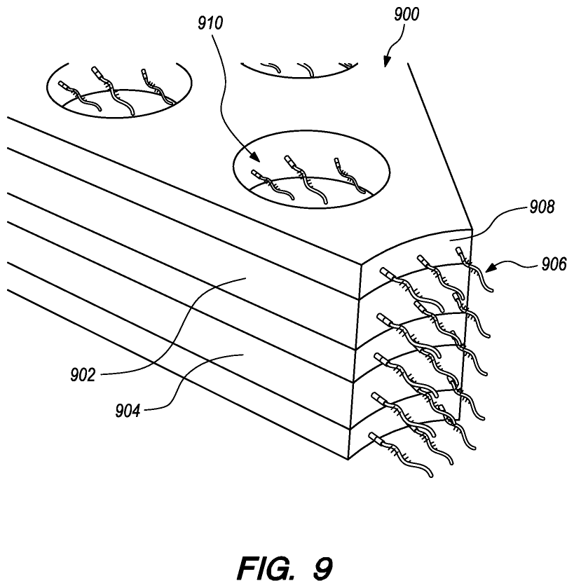

FIGS. 9 to 11 schematically depict hybridization of target (e.g., SARS-CoV-2 specific) oligonucleotides to oligonucleotide probes designed to be complementary thereto according to some embodiments. Hybridization of target oligonucleotides to oligonucleotide probes is a part of methods for detecting and quantifying the target oligonucleotides according to some embodiments, such as those described in U.S. Utility patent application Ser. No. 16/237,570, the contents of which have been previously incorporated by reference herein.

FIG. 9 schematically depicts a portion of a functionalized nanochannel array 900 with nanoelectrodes 902, 904 embedded therein according to some embodiments. Exemplary nanochannel arrays with nanoelectrodes embedded therein are described in U.S. Provisional Patent Application Ser. Nos. 62/711,234, 62/874,766 and 62/972,415, and U.S. Utility patent application Ser. No. 16/524,033, the contents of which have been previously incorporated by reference herein. The nanochannel array 900 also includes DNA probes 906 attached to a functionalized inner surface 908 of a nanochannel. The nanochannel array 900 further includes other DNA probes 910, which can be the same as or different from DNA probes 906, attached to inner surfaces of other nanochannels. The DNA probes 908, 910 may have the same or different sequences as described herein such as oligonucleotide probes for detecting SARS-CoV-2.

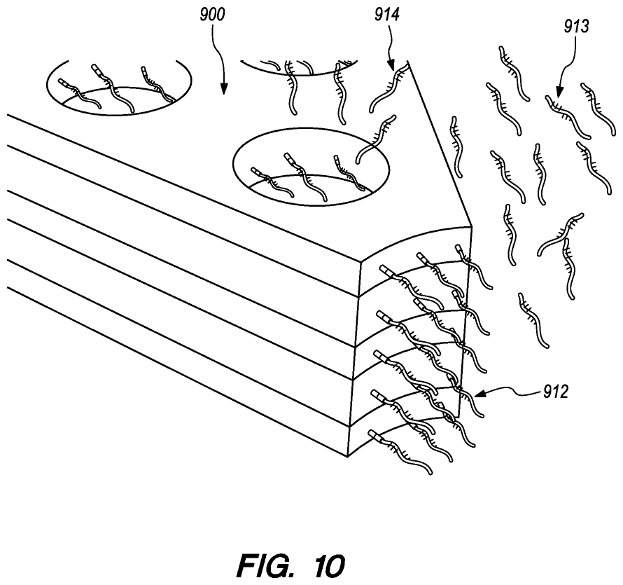

FIG. 10 schematically depicts the portion of the functionalized nanochannel array 900 depicted in FIG. 9 after extracted target SARS-CoV-2 RNA and/or cDNA molecules 912 have been added to the functionalized nanochannel array 900. FIG. 10 also depicts other extracted target SARS-CoV-2 RNA and/or cDNA molecules 914, which have also been added to the functionalized nanochannel array 900 and sensed by other nanochannels. The extracted target SARS-CoV-2 RNA and/or cDNA molecules 912, 914 may have the same or different sequences as described herein.

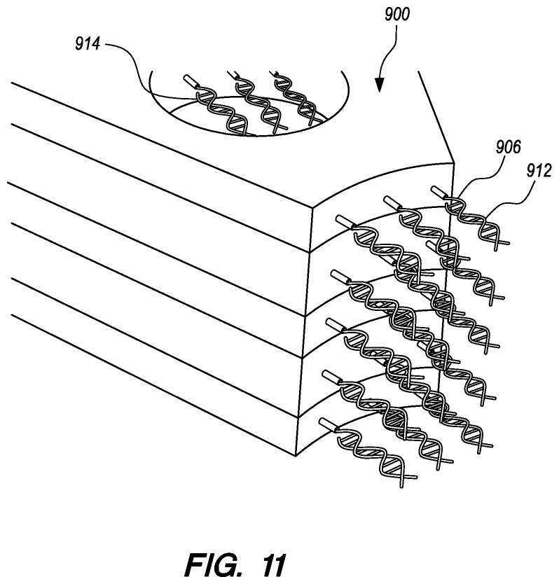

FIG. 11 schematically depicts the portion of the functionalized nanochannel array 900 depicted in FIGS. 9 and 10 after extracted target SARS-CoV-2 RNA and/or cDNA molecules 912 have attached to the SARS-CoV-2 specific oligonucleotide probes 906 coupled to a nanochannel in the functionalized nanochannel array 900. FIG. 11 also depicts other extracted target SARS-CoV-2 RNA and/or cDNA molecules 914, which have also attached to other SARS-CoV-2 specific oligonucleotide probes coupled to other nanochannels in the functionalized nanochannel array 900. The extracted target SARS-CoV-2 RNA and/or cDNA molecules 912, 914 may have the same or different sequences as described herein. After the extracted target SARS-CoV-2 RNA and/or cDNA molecules 912, 914 have attached to the SARS-CoV-2 specific oligonucleotide probes in the nanochannel array 900, electrical signals can be sensed with nanoelectrodes 902, 904 (see FIG. 9) and analyzed to detect and quantify the SARS-CoV-2 RNA and/or cDNA molecules 912, 914 as described herein and in methods such as those described in U.S. Utility patent application Ser. No. 16/237,570, the contents of which have been previously incorporated by reference herein.

The 3D nanochannel devices described herein can be used in point of care, bedside systems/platforms for detecting target biomolecules (e.g., RNA and/or cDNA related to COVID-19). The 3D nanochannel devices include preparation and extraction chambers and nanochannel arrays for sensing the target biomolecules. The point of care, bedside systems/platforms include processors and software operatively and communicatively coupled to the 3D nanochannel devices to control same and analyze data from same to generate diagnostic results. The communication between the processors and the 3D nanochannel devices may be wireless connections using Bluetooth connections. Each of the 3D nanochannel devices may have hundreds of nanochannels with each nanochannel having a plurality (e.g., two or four) nanoelectrodes embedded therein. The plurality of nanoelectrodes in each nanochannel provides sensing therein and increases sensitivity by decreasing the Debby lenses. The array of nanochannels increases the surface area to volume ratio of the 3D nanochannel devices and allows miniaturization of same and incorporation of same into small footprint/form factor point of care, bedside systems/platforms for detecting target biomolecules.

The 3D nanochannel devices described herein can detect target biomolecules without amplification (e.g., PCR) or fluorescent or other tagging, which may be used with two dimensional or planar sensors. Accordingly, the 3D nanochannel devices described herein can be used to replace amplification and tagging steps in other biochemical methods, shortening assay time.

The 3D nanochannel devices described herein have high sensitivity and have a very low detection limit in the range of 10 femtomolar concentration of target oligonucleotides or less. The 3D nanochannel devices described herein can detect a pathogen (e.g., SARS-CoV-2) and diagnose an infection (e.g., COVID-19) in a short period of time on the order of a minute to several minutes.

The 3D nanochannel devices described herein are configured for use with oropharyngeal and/or nasopharyngeal swabs to collect and process patient samples. The oropharyngeal and/or nasopharyngeal swabs with patient samples thereon are introduced into lysis buffer in the preparation and extraction chambers as described herein. Then an isothermal or gradient heating and cooling system can be used to prepare the patient sample in a solution of lysis buffer.

After the extraction step, the sample processing and analysis method using the 3D nanochannel devices described herein includes a washing step as described herein. After the washing step, a sensing step can be carried out as described herein. During the sensing step, the signal reading and intensity determination can be carried out in about one minute. The sensed signal and intensity is there processed as described herein to output qualitative and quantitative results as described herein.

In some embodiments, the 3D nanochannel devices include a first embedded nanoelectrode for sensing and second, third, and fourth nanoelectrodes for three dimensional sensing inside each nanochannel. The 3D nanochannel devices may include integrated microfluidic chambers to facilitate a washing step after sample preparation. The 3D nanochannel devices may include rechargeable batteries and may be connected to a processor via WiFi or a cloud network.

In some embodiments, a plurality of oligonucleotide probes complementary to several oligonucleotide targets indicative of a pathogen (e.g., SARS-CoV-2) or an infection (e.g., COVID-19) can be coupled to an inner surface of each nanochannel. In some embodiments, different oligonucleotide probes complementary to several oligonucleotide targets each indicative of a different pathogen or a different infection can be coupled to inner surfaces of respective nanochannels such that different nanochannels detect different pathogens and evidence of different infections. The 3D nanochannel devices described herein can be controlled by software to handle different oligonucleotide probes with different oligonucleotide targets in different nanochannels. The software can show the electrical signals sensed in different nanochannels (by different nanoelectrodes) using different colors for different numerical ranges of sensed current. Such software can analyze various sensed signals to generate intensity plots and two and three dimensional maps for observation of signals corresponding to different oligonucleotide probes inside each nanochannel.

The 3D nanochannel devices described herein can be configured to process patient samples from one patient at a time (see FIG. 6A) or from a plurality of samples from a plurality of patients (see FIG. 6B) using a plurality of isolated 3D nanochannel arrays in one system.

The probes used in the 3D nanochannel array sensors described herein may be modified to alter their surface chemistry, allowing more system control and design options. For instance, thiol modification may be used for thiol gold binding. Avidin/biotin and EDC crosslinker/N-hydroxysuccinimide (NHS) are other probe modification and target pairs that may be used with the 3D nanochannel array sensors described herein accommodating modification of structure and chemistry of immobilizing techniques.

The corresponding structures, materials, acts and equivalents of all means or step plus function elements in the claims below are intended to include any structures, materials, acts and equivalents for performing the function in combination with other claimed elements as specifically claimed. It is to be understood that while the invention has been described in conjunction with the above embodiments, the foregoing description and claims are not to limit the scope of the invention. Other aspects, advantages and modifications within the scope of the invention will be apparent to those skilled in the art to which the invention pertains.

In some embodiment the device and system use CRISPR enzymes as an immobilized probe in such structure and system to detect the target oligonucleotide within the particular pathogen, for instance using dCAS 9 or other crisper enzyme families for such platforms and systems.

In some embodiment using the provided temperature gradient can be used for performing Realtime PCR for amplification of a target molecule in such systems and platforms.

In some embodiment using such system and platform for a simple PCR protocol for amplification of the target molecule before sensing it.

In some embodiment using isothermal PCR before sensing the target molecule in an all in one system and platform described herein.

In some embodiment using monoclonal antibody as a functionalized probe to detect an antigen inside the collected sample.

In some embodiment DNA probes can be addressed by plurality of nanoelectrodes into particular nanochannel. Where the first electrode is for the addressing and the second and third electrode is for sensing the target RNA, or DNA or antigen.

In some embodiments using dressed polymer for covering the surface of the nanochannel before probe functionalization and cleaning it after sensing protocol and reusing such a sensor for diagnosis again.

A Sequence Listing is filed herewith as an ASCII text file. The name of the Sequence Listing ASCII text file is “US17378167_ST25.txt”. The date of creation of the Sequence Listing ASCII text file is Oct. 5, 2021. The size of the Sequence Listing ASCII text file is 2 KB. The Sequence Listing filed herewith is fully incorporated-by-reference herein as though set forth in full.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art, each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. Other details of the present invention may be appreciated in connection with the above-referenced patents and publications as well as generally known or appreciated by those with skill in the art. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

What is claimed is:

1. A method of detecting a pathogen in a patient sample, comprising:
providing a 3D nanochannel device having top and bottom chambers, and a 3D nanochannel array disposed in the top and bottom chambers such that the top and bottom chambers are fluidly coupled by a plurality of nanochannels in the 3D nanochannel array;
functionalizing the 3D nanochannel array by coupling an oligonucleotide probe to an inner surface of the 3D nanochannel device defining the nanochannel, wherein the oligonucleotide probe is complementary to an oligonucleotide characteristic of the pathogen;
adding a lysis buffer to the top chamber;
adding the patient sample to the lysis buffer;
extracting an oligonucleotide from the patient sample in the lysis buffer to form a sample solution;
placing top and bottom electrodes in the top and bottom chambers respectively;
applying an electrophoretic bias between the top and bottom electrodes;
applying a selection bias across first and second gating nanoelectrodes in the 3D nanochannel device to direct flow of the oligonucleotide through a nanochannel of the plurality of nanochannels;
applying a sensing bias through a sensing nanoelectrode in the 3D nanochannel device;
detecting an output current from the sensing nanoelectrode; and
analyzing the output current from the sensing nanoelectrode to detect the oligonucleotide.

2. The method of claim 1, wherein functionalizing the 3D nanochannel array by coupling the oligonucleotide probe to the inner surface of the 3D nanochannel device defining the nanochannel comprises:
adding a solution of the oligonucleotide probe to the 3D nanochannel array;
running a current through the 3D nanochannel array;
washing the 3D nanochannel array; and
reading a signal from the 3D nanochannel array to confirm functionalization of same.

3. The method of claim 2, wherein washing the 3D nanochannel array comprises using a microfluidic chamber.

4. The method of claim 1, wherein analyzing the output current from the sensing nanoelectrode to detect the oligonucleotide is performed by a processor coupled to the 3D nanochannel device.

5. The method of claim 4, wherein the processor is coupled to the 3D nanochannel device via a wired connection.

6. The method of claim 4, wherein the processor is coupled to the 3D nanochannel device via a wireless connection.

7. The method of claim 1, wherein detecting the 3D nanochannel device has more than 100 nanochannels therein.

8. The method of claim 1, wherein the 3D nanochannel device comprises first, second, third, and fourth nanoelectrodes.

9. The method of claim 8, wherein the first nanoelectrode is configured for sensing, and
wherein the second, third, and fourth nanoelectrodes are configured for three dimensional sensing.

10. The method of claim 1, wherein extracting the oligonucleotide from the patient sample in the lysis buffer to form the sample solution comprises heating the lysis buffer with the patient sample therein.

11. The method of claim 1, further comprising displaying a qualitative result.

12. The method of claim 1, further comprising displaying a quantitative result.

13. The method of claim 1, wherein the 3D nanochannel device comprises a battery.

14. The method of claim 1, wherein the method can be carried out in a point of care, bedside system.

15. The method of claim 1, wherein the 3D nanochannel array increases the surface area to volume ratio of the 3D nanochannel device.

16. The method of claim 1, wherein the method is configured to detect the oligonucleotide at a 10 femtomolar concentration or less.

17. The method of claim 1, wherein the method is configured to detect the oligonucleotide in about one minute.

18. The method of claim 1, further comprising functionalizing the 3D nanochannel array by coupling a second oligonucleotide probe to an inner surface of the 3D nanochannel device defining a second nanochannel,
wherein the second oligonucleotide probe is different from the oligonucleotide probe, and
wherein the second oligonucleotide probe is complementary to a second oligonucleotide.

19. The method of claim 18, wherein the second oligonucleotide is characteristic of the pathogen.

20. The method of claim 18, wherein the second oligonucleotide is characteristic of another pathogen.

21. The method of claim 18, further comprising displaying first and second colors corresponding to number ranges for the oligonucleotide probe and the second oligonucleotide probe respectively.

22. The method of claim 18, further comprising displaying first and second plots corresponding to number ranges for the oligonucleotide probe and the second oligonucleotide probe respectively.

23. The method of claim 1, wherein adding the patient sample to the lysis buffer comprises a swab with the patient sample thereof into the lysis buffer.

24. The method of claim 23, further comprises processing a single swab from a single patient.

25. The method of claim 23, further comprises processing a plurality of swabs from a plurality of patients using a plurality of 3D nanochannel arrays.

26. The method of claim 1, wherein extracting the oligonucleotide from the patient sample comprises heating the lysis buffer to about 98° C. to about 100° C.

27. The method of claim 26, further comprising cooling the lysis buffer before applying the electrophoretic bias between the top and bottom electrodes.

28. The method of claim 1, further comprising performing target genome sequencing using end-to-end barcode oligonucleotides or components thereof, which can be aligned on the inner surface defining the nanochannel and read.

29.-52. (canceled)
